(19) United States
(12) Patent Application Publication
(10) Pub. No.: US 2004/0141940 A1
(43) Pub. Date: Jul. 22, 2004

(54) Silica/alumina composite filler useful for mattifying skin

(75) Inventor: Guillaume Cassin, Villebon Sur Yvette (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 10/698,403

(22) Filed: Nov. 3, 2003

Related U.S. Application Data
(60) Provisional application No. 60/428,741, filed on Nov. 25, 2002.

(30) Foreign Application Priority Data
Nov. 12, 2002 (FR) 0214117

(51) Int. Cl. A61K 7/06; A61K 7/11

(52) U.S. Cl. 424/70.16; 424/401

(57) ABSTRACT

The present invention relates to a silica/alumina composite filler and its uses, particularly for greasy skin and skin to be mattified.

### REFERENCE TO PRIOR APPLICATIONS

This application claims priority to U.S. provisional application 60/428,741 filed Nov. 25, 2002, and to French patent application 0214117 filed Nov. 12, 2002, both incorporated herein by reference.

### FIELD OF THE INVENTION

The present invention relates to the use of a dispersion of colloidal particles of silica/alumina composite filler in a composition suited for topical application to the skin. In a preferred embodiment the dispersion acts as a mattifying agent.

It also relates to a process for the treatment of greasy skin, comprising the topical application to the skin of a composition comprising, in a physiologically acceptable medium, a dispersion of colloidal particles of at least one silica/alumina composite filler and, optionally, at least one thickening hydrocolloid.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

The shininess of the skin, often related to significant secretion of sebum, is a problem which more particularly affects adolescents but which can also be displayed by adults under the effect in particular of hyperproduction of androgens. It can also be related to the sweat resulting from physical activity or weather conditions. In point of fact, a shiny skin is regarded as unattractive, all the more so since it often results in poorer performance of the makeup, which has a tendency to visually deteriorate over the course of the day.

Conventional “mattifying” compositions generally comprise powders which absorb sebum and the excess oil of the composition not absorbed by the skin. Mention may in particular be made, among mattifying powders of natural or synthetic origin, of fillers, such as talc, starch, mica, silica, nylon powders, polyethylene powders, poly-β-alanine or poly(methyl) (meth)acrylate powders. Fillers of this type exhibit the disadvantage of giving an unnatural powdery appearance to the skin which can even accentuate skin defects. Furthermore, the compositions comprising them are generally desiccating in the long term and are difficult to spread. Their mattifying effect is not very long-lasting. Finally, several of these fillers have a tendency to sediment, unless they are formulated in relatively viscous compositions.

In addition, provision has been made to use, as mattifying agents, vinylpyrrolidone/1-triacontene copolymers (FR-2 820 972) or polytetrafluoroethylene (FR-2 820 977), styrene-acrylic (FR-2 801 215) or melamine-formaldehyde or urea-formaldehyde (FR-2 792 642) resin particles. These mattifying agents confer a degree of opaqueness on the compositions comprising them, whereas transparent compositions are increasingly desired by consumers.

Also disclosed, in Application EP-0 682 939, are mattifying agents composed of colloidal dispersions of inorganic particles, in particular silica, which can be prepared by a sol-gel process. These mattifying agents certainly make it possible to prepare transparent compositions but they exhibit the disadvantage of destabilizing the compositions comprising them in the presence of a thickening hydrocolloid, in particular at acidic pH.

In point of fact, it is often useful to make provision for thickeners in cosmetic compositions in order to obtain textures which are easy to apply and which are pleasant to the touch. In particular, the use of water-soluble or water-dispersible thickeners is particularly useful in products for greasy skin, which are formulated based on water in order not to accentuate the shininess of the skin and at an acidic pH close to that of the skin in order to provide optimum tolerance.

### DETAILED DESCRIPTION OF THE PREFERRED EMBODIMENTS

The inventor has now discovered that the use of an aqueous dispersion of a silica/alumina composite filler makes it possible to formulate products for greasy skin which have a good mattifying power while offering good physiological compatibility in terms of pH and good cosmetic quality in the sense that these products can for example be found in the form of gelled transparent compositions which retain their appearance for several years.

These composite fillers have been disclosed in particular in U.S. Pat. No. 5,118,727 as binding agent for the manufacture of ceramic moulds intended for the production of metal components and in U.S. Pat. No. 2,892,797 in compositions such as paint emulsions, catalysts, rubbers or compositions for the treatment of textiles. They have also been used by the present assignee for smoothing wrinkles and fine lines by a tightening effect (unpublished application).

They have also been disclosed in Application WO 90/01919 as starting material for the encapsulation of polymers intended for the artificial colouring of the skin. In this publication, the composite silica sol solution used is gelled by addition of calcium chloride, so that it is no longer found in the form of a colloidal dispersion.

These documents do not suggest the use of a colloidal dispersion of silica/alumina composite particles for caring for greasy skin, or for mattifying skin.

For its part, Application FR-2 167 931 discloses a composition for caring for the skin, in particular with an antiwrinkle effect, comprising a protein derived from keratins and a colloidal dispersion of silica which can be a silica/alumina composite. When it is used on greasy skin, this composition is rinsed after application, in order to remove the emulsified sebum and the whitish deposit formed by the silica on the skin, and then a second coat is applied.

However, the composition disclosed in this document does not include thickening hydrocolloid within the meaning of the present invention. It therefore cannot be predicted, on reading this document, that silica/alumina composite colloidal dispersions can make it possible to formulate compositions which are stable in the presence of these hydrocolloids.

One preferred embodiment of the invention is a process for mattifying skin, comprising applying to skin to be mattified a mattifying-effective amount of a dispersion of colloidal particles of silica/alumina composite filer in a composition suitable for topical application to the skin.

Another preferred subject of the present invention is a process for the treatment of greasy skin comprising the topical application, to the skin, of a composition comprising, in a physiologically acceptable medium, a dispersion of colloidal particles of at least one silica/alumina composite filler and at least one thickening hydrocolloid selected from the group consisting of: carboxyvinyl polymers which may or may not be modified; polyacrylates and polymethacrylates; polyacrylamides; polymers and copolymers of 2-acrylamido-2-methylpropanesulphonic acid which are optionally crosslinked and/or neutralized and/or rendered hydrophobic by grafting; crosslinked anionic copolymers of acrylamide and of 2-acrylamido-2-methylpropanesulphonic acid; and polysaccharide biopolymers.

The invention also relates to the use, as mattifying agent, of a dispersion of colloidal particles of silica/alumina composite filler in a composition suited to topical application to the skin.

The term “colloidal particles” within the meaning of the present invention is understood to mean a dispersion of particles having a number-average diameter of between 3 and 150 nm, preferably between 5 and 30 nm, better still between 10 and 15 nm. These particles retain the abovementioned diameters in the composition comprising them, without aggregating, and therefore do not have thickening properties in the sense that a composition composed of 15% by weight of colloidal particles according to the invention in water exhibits a viscosity of less than 0.05 Pa.s for a shear rate equal to 10⁻¹ s, the viscosity being measured at 25° C. using a RheoStress RS150 rheometer from Haake in cone/plate configuration, the cone having a diameter of 60 mm and an angle of 2°. Furthermore, the dispersions of particles according to the invention are transparent in the sense that the turbidity of compositions composed of particles at 1% by weight in water is less than 200 NTU, the measurement being carried out at 25° C. using a Hach 2100P turbidimeter. This property of the particles of forming a colloidal solution is related to their zeta potential, which is preferably less than −20 mV and more preferably less than −25 mV at pH 7 and at 25° C., as measured using a Delsa 440SX device from Coulter Scientific Instruments.

The term “particles of silica/alumina composite filler” within the meaning of the present invention is understood to mean particles composed of silicon oxide and with a surface which has been chemically modified so as to replace at least some of the silicon atoms by aluminium atoms, forming at most a monomolecular layer of aluminium. The surface portion of these particles which is covered with aluminium is generally between 1 and 100%, preferably between 1 and 10%, better still between 4 and 6%. In one preferred embodiment the particles are core/shell particles with a silica core, and between 4 and 6% of the surface area of the core is covered with aluminium.

These particles may be prepared as disclosed in U.S. Pat. No. 2,892,797, by mixing a silica sol with a sodium aluminate. Furthermore, they are commercially available from Grace under the commercial reference Ludox AM® in the form of aqueous dispersions comprising 30% by weight of active material.

The composition according to the invention is suited to topical application to the skin and therefore generally comprises a physiologically acceptable medium, that is to say a medium compatible with the skin and/or its superficial body growths. Thus, the composition according to the invention preferably has a pH of less than 7, better still of less than 6, the pH being preferably above 4, more preferably above 5. According to a preferred embodiment, the pH of the composition is between 5 and 6.

The amount of particles of composite filler present in the composition can vary to a large extent depending on the desired effect. By way of example, these particles can represent (as active material) from 0.01 to 10% by weight and preferably from 0.1 to 5% by weight, with respect to the total weight of the composition.

As indicated above, in addition to the colloidal dispersion of silica/alumina composite, the composition according may include at least one thickening hydrocolloid.

The term “thickening hydrocolloids” is understood to mean water-soluble or water-dispersible polymers which comprise at least 20 monomer units and which, at a concentration of 0.2% by weight in an aqueous, alcoholic or aqueous/alcoholic medium, confer on this medium a viscosity of greater than 5 mPa.s for a shear rate equal to 10 s⁻¹, the measurement being carried out at 25° C. using a RheoStress RS2150 rheometer from Haake in cone/plate configuration, the measuring cone having a diameter of 60 mm and an angle of 2°. The polymers in question generally have a number-average molecular weight ranging from 1 000 to 20 000 000 g/mol, preferably 20 000 to 10 000 000 g/mol and more preferably still from 100 000 to 800 000 g/mol.

Examples of such hydrocolloids which can be used in the composition according to the invention include: carboxyvinyl polymers which may or may not be modified, such as the products sold under the names Carbopol (CTFA name: carbomer) and Pemulen (CTFA name: Acrylates/C₁₀₋₃₀ alkyl acrylate crosspolymer) by Goodrich; polyacrylates and polymethacrylates, such as the products sold under the names of Lubrajel and Norgel by Guardian or under the name Hispagel by Hispano Chimica; polyacrylamides; polymers and copolymers of 2-acrylamido-2-methylpropanesulphonic acid which are optionally crosslinked and/or neutralized and/or rendered hydrophobic by grafting, such as the poly(2-acrylamido-2-methylpropanesulphonic acid) solid by Clariant under the name “Hostacerin AMPS” (CTFA name: ammonium polyacryldimethyltauramide); crosslinked anionic copolymers of acrylamide and of 2-acrylamido-2-methylpropanesulphonic acid (AMPS) which are provided, for example, in the form of a W/O emulsion, such as those sold under the name Sepigel 305 (CTFA name: Polyacrylamide/C13-14 isoparaffin/Laureth-7) and under the name Simulgel 600 (CTFA name: Acrylamide/Sodium acryloyldimethyltaurate copolymer/Isohexadecane/Polysorbate 80) by Seppic; polysaccharide biopolymers, such as xanthan gum, guar gum, locust bean gum, acacia gum, scleroglucans, chitin and chitosan derivatives, carrageenans, gellans, alginates or celluloses, such as microcrystalline cellulose, carboxymethylcellulose, hydroxymethyl-cellulose and hydroxypropylcellulose; and their mixtures.

The amount of hydrocolloid is not limited. Preferably the composition according to the invention can, for example, comprise from 0.1 to 10% and better still from 0.2 to 6% of one or more such hydrocolloids, with respect to the total weight of the composition.

The composition according to the invention can be provided in any form, for example those forms conventionally used for topical application and in particular in the form of aqueous gels. It can also, by addition of a fatty or oily phase, be provided in the form of dispersions or of emulsions with a liquid or semi-liquid consistency of the milk type, obtained by dispersion of fatty phase in an aqueous phase (O/W) or vice verse (W/O), or of suspensions or emulsions with a soft, a semi-solid or solid consistency of the cream or gel type, or of multiple emulsions (W/O/W or O/W/O), of microemulsions, of vesicular dispersions of ionic and/or nonionic type, or of wax/aqueous phase dispersions. These compositions are prepared according to the usual methods.

According a preferred embodiment of the invention, the composition is provided in the form of an oil-in-water emulsion or of an aqueous gel.

In the case where it is formulated in the form of an emulsion, the proportion of the oily phase of the emulsion can range, for example, from 0.5 to 20% by weight, with respect to the total weight of the composition. The oils, the emulsifiers and the coemulsifiers used in the composition in the emulsion form are selected from the group consisting of those conventionally used in the cosmetics or dermatological field. The emulsifer and the coemulsifier are generally present in the composition in a proportion ranging from 0.3 to 30% by weight and preferably from 0.5 to 20% by weight, with respect to the total weight of the composition.

In the case where it is in the form of an O/W emulsion, the composition according to the invention can comprise, as surfactants, at least one compound selected from the group consisting of: esters of polyols and of fatty acids comprising a saturated or unsaturated chain comprising, for example, from 8 to 24 carbon atoms and better still from 12 to 22 carbon atoms, and their oxyalkylated derivatives, that is to say comprising oxyethylene and/or oxypropylene units, such as esters of glycerol and of C₈-C₂₄ fatty acids, and their oxyethylenated derivatives, esters of sorbitol and of C₈-C₂₄ fatty acids, and their oxyalkylenated derivatives, esters of sugars (sucrose, glucose, alkylglucose) and of C₈-C₂₄ fatty acids, and their oxyalkylenated derivatives; esters of polyethylene glycol and of C₈-C₂₄ fatty acids, and their oxyalkylenated derivatives; ethers of polyalkylene glycol and of C₈-C₂₄ fatty alcohols; the ethers of sugars and of C₈-C₂₄ fatty alcohols; and their mixtures.

In an alternative form, the composition according to the invention in the emulsion form can include an ionic amphiphilic polymer and be devoid of emulsifier.

The composition according to the invention can also comprise the adjuvants, for example those conventional in the cosmetics and dermatological fields, such as hydrophilic or lipophilic gelling agents, active principles, preservatives, solvents, fragrances, fillers, pigments, odour absorbers and colouring materials. The amounts of these various adjuvants may be those conventionally used in the fields under consideration, for example from 0.01 to 20% of the total weight of the composition. These adjuvants, depending on their nature, can be introduced into the fatty phase or into the aqueous phase. These adjuvants, and their concentrations, preferably are such that they are not harmful to the advantageous properties of the composite filler according to the invention.

The composition according to the invention will preferably comprise, as active principles, at least one active principle selected from the group consisting of: retinoids and in particular retinol; zinc salts, such as zinc gluconate; a Laminaria saccharina extract; a wild yam extract; triclosan; phenoxyethanol; octoxyglycerin; octanoylglycine; a clove extract; caprylyl glycol; azelaic acid; α-hydroxy acids, such as lactic acid or glycolic acid; β-hydroxy acids, in particular salicylic acid and its derivatives, such as 5-(n-octanoyl)salicylic acid; ursolic acid; panthenol; niacinamide and octopirox.

Mention may be made, as fillers, for example, of polyamide (Nylon) particles and in particular those sold under the names Orgasol by Atochem; polyethylene powders; microspheres based on acrylic copolymers, such as those made of ethylene glycol dimethacrylate/lauryl methacrylate copolymer sold by Dow Corning under the name Polytrap; poly(methyl methacrylate) microspheres sold under the name Microsphere M-100 by Matsumoto or under the name Covabead LH85 by Wacker; powders formed of ethylene/acrylate copolymer, such as those sold under the name Flobeads by Sumitomo Seika Chemicals; expanded powders, such as hollow microspheres and in particular the microspheres formed from a terpolymer of vinylidene chloride, of acrylonitrile and of methacrylate sold under the name Expancel by Kemanord Plast under the references 551 DE 12 (particle size of approximately 12 μm and density 40 kg/m³), 551 DE 20 (particle size of approximately 30 μm and density 65 kg/m³) or 551 DE 50 (particle size of approximately 40 μm), or the microspheres sold under the name Micropearl F 80 ED by Matsumoto; powders formed from natural organic materials, such as powders formed from starch, in particular from crosslinked or noncrosslinked maize, wheat or rice starches, such as the powders from starch crosslinked by octenylsuccinic anhydride sold under the name Dry-Flo by National Starch; polyamide fibres; silicone resin microbeads, such as those sold under the name Tospearl by Toshiba Silicone, in particular Tospearl 240; silica; metal oxides, such as titanium dioxide, zinc oxide or alumina; mica; talc; sericite; boron nitride; clays; and their mixtures.

The invention will now be illustrated by the following nonlimiting examples. In these examples, the amounts are indicated as percentage by weight.

### EXAMPLES

### Comparative Example 1

### Cosmetic Composition

| | |
|---|---|
| Water | 80.42 g |
| Glycerol | 1.00 g |
| Phenoxyethanol | 1.00 g |
| Lactic acid | 0.06 g |
| Gellan | 0.20 g |
| Ethanol | 10.00 g |
| Dispersion of silica colloidal particles | 7.32 g |
| (Cosmo S40 from Catalyst and Chemicals)* | |

*aqueous dispersion, comprising 41% of active material of silica having a particle size of 18 nm

### Comparative Example 2

### Cosmetic Composition

| | |
|---|---|
| Water | 80.02 g |
| Glycerol | 1.00 g |
| Phenoxyethanol | 1.00 g |
| Lactic acid | 0.06 g |
| Ethanol | 10.00 g |
| Dispersion of silica colloidal particles | 7.32 g |
| (Cosmo S40 from Catalyst and Chemicals)* | |
| Carboxyvinyl polymer (Carbopol 980) | 0.30 g |
| Triethanolamine | 0.30 g |

*aqueous dispersion, comprising 41% of active material of silica having a particle size of 18 nm

### Example 1

### Cosmetic Composition

| | |
|---|---|
| Water | 77.74 g |
| Glycerol | 1.00 g |
| Phenoxyethanol | 1.00 g |
| Lactic acid | 0.06 g |
| Ethanol | 10.00 g |
| Gellan | 0.20 g |
| Dispersion of composite colloidal particles | 10.00 g |
| of silica/alumina (Ludox AM from Grace) | |

### Example 2

### Cosmetic Composition

| | |
|---|---|
| Water | 77.34 g |
| Glycerol | 1.00 g |
| Phenoxyethanol | 1.00 g |
| Lactic acid | 0.06 g |
| Ethanol | 10.00 g |
| Dispersion of composite colloidal particles | 10.00 g |
| of silica/alumina (Ludox AM from Grace) | |
| Carboxyvinyl polymer (Carbopol 980) | 0.30 g |
| Triethanolamine | 0.30 g |

### Example 3

### Cosmetic Composition

| | |
|---|---|
| Water | 63.82 g |
| Polyglyceryl stearate | 1.00 g |
| Oxyethylenated sorbitan monostearate | 0.75 g |
| Disodium salt of stearoylglutamate | 0.50 g |
| Isocetyl stearate | 5.00 g |
| Caprylic/capric triglycerides | 2.00 g |
| Isononyl isononanoate | 3.00 g |
| Butylparaben | 0.25 g |
| Chlorophenesin | 0.25 g |
| Lactic acid | 0.10 g |
| Dispersion of composite colloidal particles | 23.33 g |
| of silica/alumina (Ludox AN from Grace) | |

### Example 4

### Demonstration of the Mattifying Effect of the Compositions According to the Invention

The compositions of Examples 1 and 3 were tested on a panel of seven women having greasy skin and exhibiting cutaneous microrelief irregularities. An immediate decrease in the shininess of the skin was observed after application of these compositions.

### Example 5

### Stability Test

The stability of the compositions of Comparative Examples 1 and 2 and of Examples 1 to 3 above was compared after storage for two months at 4° C., 25° C., 37° C. and 45° C.

A composition was regarded as stable if all the macroscopic characteristics (colour, smell, distribution of its phases, viscosity, pH) and microscopic characteristics of the composition remained unchanged. It was regarded as unstable if at least one of these characteristics was modified.

The results are collated in Table 1 below:

TABLE 1
Stability test

| Composition | pH | Stability |
|---|---|---|
| Comparative Example 1 | 5.7 | unstable |
| (comprising 3% of silica) | | |
| Comparative Example 2 | 6.7 | unstable |
| (comprising 3% of silica) | | |
| Example 1 | 5.1 | stable |
| (comprising 3% of composite silica) | | |
| Example 2 | 6.3 | stable |
| (comprising 3% of composite silica) | | |
| Example 3 | 6.8 | stable |
| (comprising 7% of composite silica) | | |

It emerges from the above Table 1 that preferred compositions according to the invention, which comprise silica/alumina composite particles, are stable at a pH of less than 7, and even of less than 6, including in the presence of thickening polymers, whereas such is not the case with the compositions including colloidal particles of silica not in the composite form, which have a tendency to set solid after storage for one month.

The above written description of the invention provides a manner and process of making and using it such that any person skilled in this art is enabled to make and use the same, this enablement being provided in particular for the subject matter of the appended claims, which make up a part of the original description. Moreover, fully described and enabled is a process for the cosmetic treatment of greasy skin comprising the topical application, to the skin, of a composition comprising, in a physiologically acceptable medium, a dispersion of colloidal particles of at least one silica/alumina composite filler and at least one thickening hydrocolloid, where the hydrocolloid is preferably selected from the group consisting of: carboxyvinyl polymers which may or may not be modified; polyacrylates and polymethacrylates; polyacrylamides; polymers and copolymers of 2-acrylamido-2-methylpropanesulphonic acid which are optionally crosslinked and/or neutralized and/or rendered hydrophobic by grafting; crosslinked anionic copolymers of acrylamide and of 2-acrylamido-2-methylpropanesulphonic acid; and polysaccharide biopolymers. Other preferred embodiments of the invention similarly fully described and enabled include the cosmetic use as mattifying agent of a dispersion of colloidal particles of silica/alumina composite filer in a composition suited to topical application to the skin.

As used above, the phrase “selected from the group consisting of” includes mixtures of the specified materials.

All references, patents, applications, tests, standards, documents, publications, brochures, texts, articles, etc. mentioned herein are incorporated herein by reference. Where a numerical limit or range is stated, all values and subranges therewithin are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

1. A process for the treatment of greasy skin comprising topically applying to greasy skin a composition comprising, in a physiologically acceptable medium, a dispersion of colloidal particles of at least one silica/alumina composite filler and at least one thickening hydrocolloid, wherein the thickening hydrocolloid is selected from the group consisting of: carboxyvinyl polymers; modified carboxyvinyl polymers; polyacrylates; polymethacrylates; polyacrylamides; polymers and copolymers of 2-acrylamido-2-methylpropanesulphonic acid which are optionally crosslinked and/or neutralized and/or rendered hydrophobic by grafting; crosslinked anionic copolymers of acrylamide and of 2-acrylamido-2-methylpropanesulphonic acid; polysaccharide biopolymers, and mixtures thereof.

2. The process according to claim 1, wherein the particles have a number-average diameter of 5-30 nm.

3. The process according to claim 2, wherein the particles have a number-average diameter of 10-15 nm.

4. The process according to claim 1, wherein the particles are core/shell particles with a silica core, and wherein 4-6% of the surface area of the core is covered with aluminium.

5. The process according to claim 1, wherein the particles have a zeta potential of less than −25 mV at pH 7 and at 25° C.

6. The process according to claim 1, wherein the composition has a pH of less than 7.

7. The process according to claim 1, wherein the composition has a pH of less than 6.

8. The process according to claim 1, wherein said composition comprises at least one polysaccharide biopolymer selected from the group consisting of xanthan gum, guar gum, locust bean gum, acacia gum, scleroglucans, chitin and chitosan derivatives, carrageenans, gellans, alginates and celluloses.

9. The process according to claim 1, wherein said composition comprises at least one polysaccharide biopolymer selected from the group consisting of microcrystalline cellulose, carboxymethylcellulose, hydroxymethylcellulose and hydroxypropylcellulose.

10. The process according to claim 1, wherein the composition is in the form of an oil-in-water emulsion.

11. The process according to claim 2, wherein the composition is in the form of an oil-in-water emulsion.

12. The process according to claim 3, wherein the composition is in the form of an oil-in-water emulsion.

13. The process according to claim 4, wherein the composition is in the form of an oil-in-water emulsion.

14. The process according to claim 8, wherein the composition is in the form of an oil-in-water emulsion.

15. The process according to claim 1, wherein the composition is in the form of an aqueous gel.

16. The process according to claim 2, wherein the composition is in the form of an aqueous gel.

17. The process according to claim 3, wherein the composition is in the form of an aqueous gel.

18. The process according to claim 8, wherein the composition is in the form of an aqueous gel.

19. The process according to claim 1, wherein the composition comprises 0.1-5% by weight of colloidal particles of silica/alumina composite filler.

20. A process for mattifying skin, comprising applying to skin to be mattified a mattifying-effective amount of a dispersion of colloidal particles of silica/alumina composite filer in a composition suitable for topical application to the skin.
